(19) United States
(12) Patent Application Publication
(10) Pub. No.: US 2025/0120608 A1
(43) Pub. Date: Apr. 17, 2025

(54) SYSTEM AND METHOD FOR IDENTIFYING OPTIMIZED INTERCONNECTIONS BETWEEN LESION SEGMENTS AND COMPUTING ASSOCIATED INTERLESION DISTANCES

(71) Applicant: St. Jude Medical, Cardiology Division, Inc., St. Paul, MN (US)

(72) Inventors: Hai Huang, Woodbury, MN (US); Jayanth Sebastian Kallingal, Woodbury, MN (US); Kristi Tanouye, Minneapolis, MN (US); Aline Favilla, Plymouth, MN (US)

(21) Appl. No.: 18/912,876

(22) Filed: Oct. 11, 2024

Related U.S. Application Data
(60) Provisional application No. 63/589,722, filed on Oct. 12, 2023.

(51) Int. Cl. A61B 5/06; A61B 18/00; A61B 18/14

(52) U.S. Cl. CPC A61B 5/064; A61B 18/1492; A61B 2018/00351; A61B 2018/00577

(57) ABSTRACT

An electroanatomical mapping system identifies optimized interconnections between ablation lesion segments. The system receives lesion markers, each representing an ablation lesion segment, and defines one or more disjoint subsets thereof. For each disjoint subset, the system defines optimized interconnections between lesion markers and outputs a graphical representation of the disjoint sets and the optimized interconnections. The disjoint subsets may be defined as minimum cost spanning trees. The optimized interconnections can include the edges of the minimum cost spanning tree as well as additional cycle-closing edges that are not edges of the minimum cost spanning tree, where the cycle-closing edges are leaf edges that satisfy one or more cycle-closing criteria.

### CROSS-REFERENCE TO RELATED APPLICATIONS

This application claims the benefit of U.S. provisional application No. 63/589,722, filed 12 Oct. 2023, which is hereby incorporated by reference as though fully set forth herein.

### BACKGROUND

The present disclosure relates generally to a system and method for presenting information representative of lesion formation in tissue during an ablation procedure. In particular, the present disclosure relates to a system and method for automatically identifying optimized groupings of and interconnections between lesion segments, as represented by lesion markers, computing corresponding interlesion distances, and displaying the groupings and distances on an image or model of tissue.

When tissue is ablated, or at least subjected to ablative energy, lesions form in the tissue. It is known that such ablation therapy may be used to treat various conditions afflicting the human anatomy, such as ectopic atrial tachycardia, atrial fibrillation, atrial flutter, and other cardiac arrythmias. Arrhythmias can create a variety of dangerous conditions including irregular heart rate, loss of synchronous atrioventricular contraction, and stasis of blood flow, which can, in turn, lead to a variety of ailments.

It is believed that the primary cause of atrial arrhythmia is stray electrical signals within the left or right atrium of the heart. Thus, in some ablation therapies, an electrode or electrodes mounted on or in the ablation catheter are used to create necrosis in cardiac tissue by imparting ablative energy (e.g., radio frequency energy, cryoablation, lasers, chemicals, high-intensity focused ultrasound, etc.) to the cardiac tissue to create a lesion that disrupts undesirable electrical pathways and thereby limits or prevents stray electrical signals that can lead to arrhythmias.

Various approaches to tracking the formation of ablation lesions are known. In many instances, these approaches include the automatic and/or manual creation of lesion markers. Each such lesion marker, whether automatically or manually created, represents a segment of a lesion in the tissue. When these lesion markers are displayed on an image or model of the tissue, a lesion formation map is created that allows a clinician to visualize the lesion.

One challenge associated with interpreting a lesion formation map, however, is understanding how to interpret the distances between lesion markers on the lesion formation map (i.e., the “interlesion distances”). For instance, two lesion markers may be sufficiently close to each other that the lesion segments represented thereby can be interpreted as forming a single, continuous lesion that will likely be sufficient to disrupt errant conduction.

Alternatively, two lesion markers may represent lesion segments that have a sufficiently large gap therebetween to permit errant conduction. In this circumstance, the clinician might find it desirable to place an additional lesion segment between the two markers to eliminate the gap and form a continuous lesion.

In still other instances, two lesion markers may represent lesion segments that are so far apart that they should be interpreted as part of separate lesions.

Thus, it would be desirable for an electroanatomical mapping system and/or an ablation system to be able to identify optimized groupings of and interconnections between automatically- and/or manually-created lesion markers, compute the corresponding interlesion distances, and display the groupings and distances on an image or model of tissue to enhance a clinician's ability to treat cardiac arrhythmias through the creation of lesions that interrupt the underlying errant conduction pathways.

### BRIEF SUMMARY

The instant disclosure provides a method of identifying optimized interconnections between ablation lesion segments. The method includes: receiving, at an electroanatomical mapping system, a plurality of lesion markers, each lesion marker of the plurality of lesion markers representing an ablation lesion segment in a tissue; the electroanatomical mapping system defining one or more disjoint subsets of the plurality of lesion markers; for each disjoint subset, the electroanatomical mapping system defining one or more optimized interconnections between lesion markers included within the respective disjoint subset; and outputting on a display of the electroanatomical mapping system a graphical representation of the one or more disjoint sets and the one or more optimized interconnections between lesion markers on a graphical representation of the tissue.

In embodiments of the disclosure, the electroanatomical mapping system can define the one or more disjoint subsets of the plurality of lesion markers by defining a minimum cost spanning tree for each of the one or more disjoint subsets of the plurality of lesion markers. For example, the electroanatomical mapping system can: define a weighted, undirected graph including a plurality of vertices and a plurality of weighted edges, wherein the plurality of vertices correspond to the plurality of lesion markers and each weighted edge of the plurality of weighted edges connects a pair of vertices of the plurality of vertices; sort the plurality of weighted edges by ascending weight; and iteratively analyze each weighted edge of the sorted plurality of weighted edges, at each iteration merging a first disjoint subset containing a first vertex of the pair of vertices connected by the respective weighted edge and a second disjoint subset containing a second vertex of the pair of vertices connected by the respective weighted edge into a combined disjoint subset and adding the respective weighted edge to a minimum spanning tree of the combined disjoint subset when the weight of the respective weighted edge is below a maximum weight threshold and the first vertex and the second vertex are not in a common disjoint subset prior to being merged into the combined disjoint subset.

The method can also include, prior to iteratively analyzing the sorted plurality of weighted edges, defining a plurality of initial disjoint subsets, each initial disjoint subset including exactly one of the plurality of vertices of the graph.

It is contemplated that the weight of each weighted edge can be defined as a distance between the respective pair of vertices connected by the respective weighted edge. The distance can be a Euclidean distance.

According to aspects of the instant disclosure, the one or more optimized interconnections between lesion markers included within the respective disjoint subset can include a plurality of edges within the minimum cost spanning tree for the respective disjoint subset.

Further, the one or more optimized interconnections between lesion markers included within the respective disjoint subset can also include one or more cycle-closing edges not included within the minimum cost spanning tree for the respective disjoint subset. The electroanatomical mapping system can identify the one or more cycle-closing edges not included within the minimum cost spanning tree for the respective disjoint subset according to a series of steps including: defining a plurality of leaf edges of the minimum cost spanning tree for the respective disjoint subset; identifying a subset of the plurality of leaf edges that satisfy one or more cycle-closing criteria; and defining the subset of the plurality of leaf edges that satisfy the one or more cycle-closing criteria as the one or more cycle-closing edges not included within the minimum cost spanning tree.

In some embodiments of the disclosure, the step of identifying the subset of the plurality of leaf edges that satisfy the one or more cycle-closing criteria can include: assigning a weight to each leaf edge of the plurality of leaf edges; sorting the plurality of leaf edges by ascending weight; and iteratively analyzing each leaf edge of the sorted plurality of leaf edges with respect to each of the one or more cycle-closing criteria, at each iteration culling the respective leaf edge from the plurality of leaf edges when the respective leaf edge does not satisfy a cycle-closing criterion of the one or more cycle-closing criteria, thereby identifying the subset of the plurality of leaf edges that satisfy the one or more cycle-closing criteria.

The one or more cycle-closing criteria can include one or more of: a maximum weight threshold criterion; a cycle presence criterion; a minimum cost spanning tree path minimum weight criterion; a minimum cost spanning tree path minimum step criterion; and a concavity criterion.

Also disclosed herein is an electroanatomical mapping system including a display and a lesion segment analysis processor configured to: receive as input a plurality of lesion markers, each lesion marker of the plurality of lesion markers representing an ablation lesion segment in a tissue; define one or more disjoint subsets of the plurality of lesion markers and, for each disjoint subset, define one or more optimized interconnections between lesion markers included within the respective disjoint subset; and output on the display a graphical representation of the one or more disjoint subsets and the one or more optimized interconnections between lesion markers on a graphical representation of the tissue.

The lesion segment analysis processor can further be configured to define a minimum cost spanning tree for each of the one or more disjoint subsets of the plurality of lesion markers. For example, the lesion segment analysis processor can be configured to define the minimum cost spanning tree for each of the one or more disjoint subsets of the plurality of lesion markers by executing a series of steps including: defining a weighted, undirected graph including a plurality of vertices and a plurality of weighted edges, wherein the plurality of vertices correspond to the plurality of lesion markers and each weighted edge of the plurality of weighted edges connects a pair of vertices of the plurality of vertices; sorting the plurality of weighted edges by ascending weight; and iteratively analyzing each weighted edge of the sorted plurality of weighted edges, at each iteration merging a first disjoint subset containing a first vertex of the pair of vertices connected by the respective weighted edge and a second disjoint subset containing a second vertex of the pair of vertices connected by the respective weighted edge into a combined disjoint subset and adding the respective weighted edge to a minimum spanning tree of the combined disjoint subset when the weight of the respective weighted edge is below a maximum weight threshold and the first vertex and the second vertex are not in a common disjoint subset prior to being merged into the combined disjoint subset. The weight of each weighted edge can, in certain embodiments of the disclosure, be defined as a distance between the respective pair of vertices connected by the respective weighted edge.

The one or more optimized interconnections between lesion markers included within the respective disjoint subset can include a plurality of edges within the minimum cost spanning tree for the respective disjoint subset.

The one or more optimized interconnections between lesion markers included within the respective disjoint subset can also include one or more cycle-closing edges not included within the minimum cost spanning tree for the respective disjoint subset. In this respect, the lesion segment analysis processor can be configured to identify the one or more cycle-closing edges by executing a series of steps including: defining a plurality of leaf edges of the minimum cost spanning tree for the respective disjoint subset; identifying a subset of the plurality of leaf edges that satisfy one or more cycle-closing criteria; and defining the subset of the plurality of leaf edges that satisfy the one or more cycle-closing criteria as the one or more cycle-closing edges, wherein the one or more cycle-closing criteria includes one or more of: a maximum weight threshold criterion; a cycle presence criterion; a minimum cost spanning tree path minimum weight criterion; a minimum cost spanning tree path minimum step criterion; and a concavity criterion.

The instant disclosure also provides a method of identifying optimized interconnections between ablation lesion segments, including the following steps: receiving, at an electroanatomical mapping system, a plurality of lesion makers, each lesion marker of the plurality of lesion markers representing an ablation lesion segment in a tissue; the electroanatomical mapping system defining one or more disjoint subsets of the plurality of lesion markers; for each disjoint subset, the electroanatomical mapping system defining one or more optimized interconnections between lesion markers included within the respective disjoint subset, wherein the one or more optimized interconnections between lesion markers included within the respective disjoint subset includes: a plurality of edges included in a minimum cost spanning tree for the respective disjoint subset; and a plurality of cycle-closing edges that close cycles within the minimum cost spanning tree for the respective disjoint subset; and outputting on a display of the electroanatomical mapping system a graphical representation of the one or more disjoint subsets and the one or more optimized interconnections between lesion markers on a graphical representation of the tissue.

The plurality of cycle-closing edges can be leaf edges of the minimum cost spanning tree for the respective disjoint subset that satisfy one or more cycle-closing criteria. The one or more cycle-closing criteria can include one or more of: a maximum weight threshold criterion; a cycle presence criterion; a minimum cost spanning tree path minimum weight criterion; a minimum cost spanning tree path minimum step criterion; and a concavity criterion.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a diagrammatic view of a system for presenting information relating to lesion formation in tissue in accordance with the present teachings.

FIG. 2 is a simplified schematic diagram illustrating the electroanatomical mapping system represented in FIG. 1.

FIG. 3 is an exemplary embodiment of a display device of the system illustrated in FIG. 1 with a graphical user interface (GUI) displayed thereon.

FIG. 4 is a flowchart of representative steps that can be carried out to optimize groupings of and interconnections between lesion markers according to aspects of the instant disclosure.

FIG. 5 illustrates an exemplary set of lesion markers and representative distances therebetween.

FIG. 6 is a flowchart of representative steps that can be carried out to group lesion markers into one or more disjoint subsets according to aspects of the instant disclosure.

FIG. 7 illustrates the lesion markers of FIG. 5, grouped into two disjoint subsets according to the representative steps of FIG. 6.

FIG. 8 is a flowchart of representative steps that can be carried out to identify optimized interconnections between the lesion markers of a disjoint subset according to aspects of the instant disclosure.

FIGS. 9A through 9D illustrate optimized interconnections between lesion markers of a disjoint subset, identified according to the representative steps of FIG. 8.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

The instant disclosure provides methods and systems for identifying optimized interconnections between lesion segments and computing optimized interlesion distances. For purposes of illustration, aspects of the disclosure will be described with reference to computing interlesion distances between in cardiac tissue in conjunction with an electroanatomical mapping system, such as the EnSite Precision™ cardiac mapping system from Abbott Laboratories (Abbott Park, Illinois). Those of ordinary skill in the art will understand, however, how to apply the teachings herein to good advantage in other contexts and/or with respect to other devices.

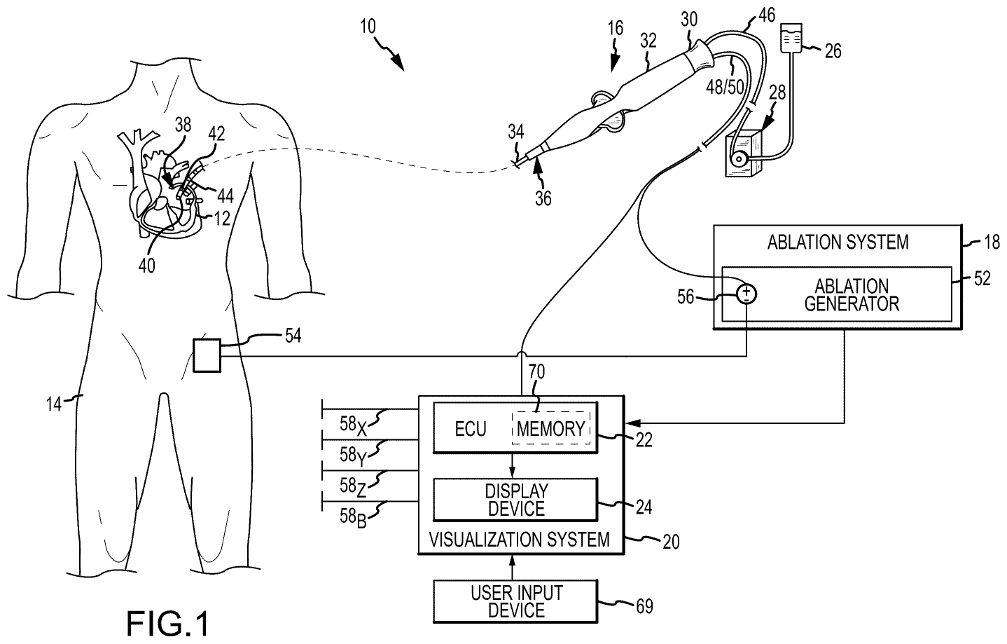

Referring now to the drawings wherein like reference numerals are used to identify identical components in the various views, FIG. 1 illustrates one exemplary embodiment of a system 10 for performing one more diagnostic and/or therapeutic functions that includes components for presenting information representative of lesion formation in a tissue 12 of a body 14 during an ablation procedure performed thereon. In an exemplary embodiment, the tissue 12 comprises heart or cardiac tissue within a human body 14. It should be understood, however, that the system 10 may find application in connection with the ablation of a variety of other tissues within human and non-human bodies.

Among other components, the system 10 includes a medical device (for example, a catheter 16), an ablation system 18, and an electroanatomical mapping system 20 for the visualization, navigation, and/or mapping of internal body structures. System 20 may include, without limitation, an electronic control unit (ECU) 22 and a display 24. Alternatively, ECU 22 and/or display 24 may be separate and distinct from, but electrically connected to and configured for communication with, system 20.

With continued reference to FIG. 1, catheter 16 is provided for examination, diagnosis, and/or treatment of internal body tissues such as tissue 12. In an exemplary embodiment, the catheter 16 comprises an ablation catheter and, more particularly, an irrigated radiofrequency (RF) ablation catheter. It should be understood, however, that catheter 16 is not limited to an irrigated catheter or an RF ablation catheter. Rather, in other embodiments, the catheter 16 may comprise a non-irrigated catheter and/or any other type of ablation catheters (e.g., cryoablation, ultrasound, laser, irreversible electroporation, and so on).

In the exemplary embodiment wherein the catheter 16 is an irrigated RF catheter, the catheter 16 is connected to a fluid source 26 providing a biocompatible fluid such as saline through a pump 28 for irrigation.

In an exemplary embodiment, the catheter 16 is electrically connected to the ablation system 18 to allow for the delivery of RF energy. The catheter 16 may include a cable connector or interface 30, a handle 32, a shaft 34 having a proximal end 36 and a distal end 38, and one or more electrodes 40, 42 mounted in or on the shaft 34 of the catheter 16. In an exemplary embodiment, the electrodes 40, 42 are disposed at or near the distal end 38 of the shaft 34, with the electrode 40 serving as an ablation electrode disposed at the extreme distal end 38 of the shaft 34 (that is, a tip electrode), and electrode 42 serving as a positioning electrode used, for example, to localize catheter 16 via electroanatomical mapping system 20. The catheter 16 may further include other conventional components such as, for example and without limitation, a temperature sensor 44, additional electrodes (e.g., ring electrodes) and corresponding conductors or leads, or additional ablation elements, e.g., a high intensity focused ultrasound ablation element.

Connector 30 provides mechanical, fluid, and electrical connection(s) for cables 46, 48, 50 extending from pump 28, ablation system 18, and electroanatomical mapping system 20. The connector 30 may be conventional and is disposed at the proximal end 36 of the catheter 16.

Handle 32 provides a location for the clinician to hold catheter 16 and may further provide means for steering or guiding the shaft 34 within the body 14. Handle 32 may likewise be conventional and those of ordinary skill in the art will recognize that the construction of the handle 32 may vary.

Shaft 34 is an elongate, tubular, flexible member configured for movement within the body 14. The shaft 34 may support, for example and without limitation, electrodes 40, 42, associated conductors, and additional electronics used for signal processing or conditioning. The shaft 34 may also permit transport, delivery and/or removal of fluids (including irrigation fluids, cryogenic ablation fluids, and bodily fluids), medicines, and/or surgical tools or instruments. The shaft 34 may be made from materials known to those of ordinary skill in the art, such as polyurethane, PTFE, and other thermoplastic elastomers, and may define one or more lumens configured to house and/or transport electrical conductors, fluids, surgical tools, or the like.

The shaft 34 may be introduced into a blood vessel or other structure within the body 14 through a conventional introducer. The shaft 34 may then be steered or guided through the body 14 to a desired location such as the tissue 12 with guidewires or other means known in the art.

Ablation system 18 includes, for example, an ablation generator 52 and one or more ablation patch electrodes 54. Ablation generator 52 generates, delivers, and controls RF energy output by ablation catheter 16, and, in particular, tip electrode 40. Generator 52 will be familiar to those of ordinary skill in the art; for purposes of illustration, however, generator 52 may be the Ampere™ RF generator available from Abbott Laboratories (Abbott Park, Illinois).

In an exemplary embodiment, generator 52 includes an RF ablation signal source 56 configured to generate an ablation signal that is output across a pair of source connectors: a positive polarity connector SOURCE (+), which may electrically connected to the tip electrode 40 of the catheter 16, and a negative polarity connector SOURCE (−), which may be electrically connected to one or more of the patch electrodes 54. It should be understood that the term “connectors” as used herein does not imply a particular type of physical interface mechanism, but is rather broadly contemplated to represent one or more electrical nodes.

The source 56 is configured to generate a signal at a predetermined frequency in accordance with one or more user specified parameters (e.g., power, time, etc.) and under the control of various feedback sensing and control circuitry as will be familiar to those of ordinary skill in the art. The source 56 may generate a signal, for example, with a frequency of about 450 kHz or greater. The generator 52 may also monitor various parameters associated with the ablation procedure including, for example, impedance, the temperature at the distal tip of the catheter, applied ablation energy, and the position of the catheter, and provide feedback to the clinician or another component within the system 10 regarding these parameters.

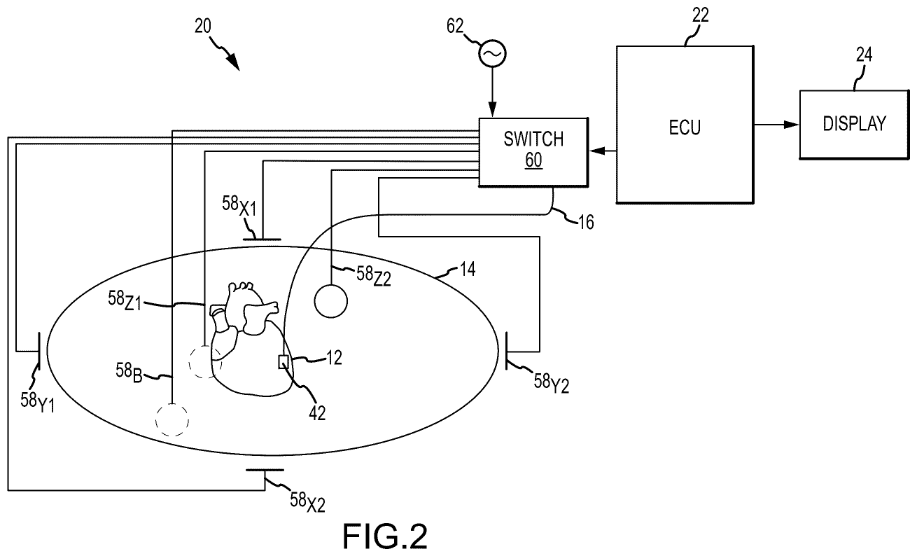

Electroanatomical mapping system 20 will be further described with reference to the schematic diagram of FIG. 2. Electroanatomical mapping system 20 can be used, for example, to create an anatomical model of the patient's heart 12 using one or more electrodes. Electroanatomical mapping system 20 can also be used to measure electrophysiology data at a plurality of points along a cardiac surface and store the measured data in association with location information for each measurement point at which the electrophysiology data was measured, for example to create a diagnostic data map of the patient's heart 12.

As one of ordinary skill in the art will recognize, electroanatomical mapping system 20 determines the location, and in some aspects the orientation, of objects, typically within a three-dimensional space, and expresses those locations as position information determined relative to at least one reference. This is referred to herein as “localization.”

For simplicity of illustration in FIG. 2, the patient 14 is depicted schematically as an oval. In the embodiment shown, three sets of surface electrodes (e.g., patch electrodes) 58_{X1}, 58_{X2}, 58_{Y1}, 58_{Y2}, 58_{Z1}, and 58_{Z2}, are shown applied to a surface of the patient 14, pairwise defining three generally orthogonal axes, referred to herein as an x-axis, a y-axis, and a z-axis. In other embodiments the patch electrodes could be positioned in other arrangements, for example multiple electrodes on a particular body surface. As a further alternative, the electrodes do not need to be on the body surface, but could be positioned internally to the body. In any case, heart 12 lies between these pairs of patch electrodes 58_{X1}, 58_{X2}, 58_{Y1}, 58_{Y2}, 58_{Z1}, and 58_{Z2}.

Each surface electrode can measure multiple signals. For example, in embodiments of the disclosure, each surface electrode can measure three resistance (impedance) signals and three reactance signals. These signals can, in turn, be grouped into three resistance/reactance signal pairs. One resistance/reactance signal pair can reflect driven values, while the other two resistance/reactance signal pairs can reflect non-drive values (e.g., measurements of the electric field generated by other driven pairs in a manner similar to that described below for electrode 42).

An additional surface reference electrode (e.g., a “belly patch”) 58_{B} provides a reference and/or ground electrode for the electroanatomical mapping system 20. The belly patch 58_{B} may be an alternative or in addition to a fixed intra-cardiac reference electrode. In alternative embodiments where electroanatomical mapping system 20 is capable of magnetic field-based localization instead of or in addition to impedance-based localization, the belly patch 58_{B} can alternatively or additionally include a magnetic patient reference sensor-anterior (“PRS-A”) positioned on the patient's chest.

It should be appreciated that patient 14 may also have most or all of the conventional electrocardiogram (“ECG” or “EKG”) system leads in place. In certain embodiments, for example, a standard set of 12 ECG leads may be utilized for sensing electrocardiograms on the patient's heart 12. This ECG information is available to electroanatomical mapping system 20 (e.g., it can be provided as input to ECU 22).

A representative catheter 16 having at least one electrode 42 is also shown. This representative catheter electrode 42 is referred to as the “roving electrode,” “moving electrode,” or “measurement electrode.” Typically, multiple electrodes 42 on catheter 16, or on multiple such catheters, will be used. In one embodiment, for example, electroanatomical mapping system 20 may utilize sixty-four electrodes on twelve catheters disposed within the heart and/or vasculature of the patient. In other embodiments, electroanatomical mapping system 20 may utilize a single catheter that includes multiple (e.g., eight) splines, each of which in turn includes multiple (e.g., eight) electrodes. The foregoing embodiments are merely exemplary, however, and any number of electrodes and/or catheters may be used.

Catheter 16 (or multiple such catheters) are typically introduced into the heart and/or vasculature of the patient via one or more introducers and using familiar procedures. Indeed, various approaches to introduce catheter 16 into a patient's heart, such as transseptal approaches, will be familiar to those of ordinary skill in the art, and therefore need not be further described herein.

Since each electrode 42 lies within the patient, location data may be collected simultaneously for each electrode 42 by electroanatomical mapping system 42. Similarly, each electrode 42 can be used to gather electrophysiological data from the cardiac surface (e.g., endocardial electrograms). The ordinarily skilled artisan will be familiar with various modalities for the acquisition and processing of electrophysiology data points (including, for example, both contact and non-contact electrophysiological mapping), such that further discussion thereof is not necessary to the understanding of the techniques disclosed herein. Likewise, various techniques familiar in the art can be used to generate graphical representations of cardiac geometry and/or cardiac electrical activity from the plurality of electrophysiology data points. Moreover, insofar as the ordinarily skilled artisan will appreciate how to create electrophysiology maps from electrophysiology data points, the aspects thereof will only be described herein to the extent necessary to understand the present disclosure.

In some embodiments, an optional fixed reference electrode (e.g., attached to a wall of the heart 12) is provided on a second intra-cardiac catheter. For calibration purposes, this electrode may be stationary (e.g., attached to or near the wall of the heart) or disposed in a fixed spatial relationship with the roving electrodes (e.g., electrodes 42), and thus may be referred to as a “navigational reference” or “local reference.” Such a fixed reference electrode may be used in addition or alternatively to the belly patch 58_{B} described above. In many instances, a coronary sinus electrode or other fixed electrode in the heart 12 can be used as a reference for measuring voltages and displacements; that is, as described below, it may define the origin of a coordinate system.

The surface electrodes are coupled to a multiplex switch 60, and the pairs of surface electrodes are selected by software running on ECU 22, which couples the surface electrodes to a signal generator 62. Alternately, switch 60 may be eliminated and multiple (e.g., three) instances of signal generator 62 may be provided, one for each measurement axis (that is, each surface electrode pairing).

ECU 22 may include a programmable microprocessor or microcontroller or may include an application specific integrated circuit (ASIC). ECU 22 include one or more central processing units (CPUs) and an input/output (I/O) interface through which ECU 22 may receive a plurality of input signals including, for example, signals generated by patch electrodes 58, the positioning electrode 42, and the ablation system 18, and generate a plurality of output signals including, for example, those used to control and/or provide data to treatment devices, the display device 24, and the switch 60. ECU 22 may be configured to perform various functions, such as those described in greater detail below, with appropriate programming instructions or code (i.e., software). Accordingly, ECU 22 may be programmed with one or more computer programs encoded on a computer storage medium for performing the functionality described herein.

Generally, three nominally orthogonal electric fields are generated by a series of driven and sensed electric dipoles (e.g., surface electrode pairs 58_{X1}/58_{X2}, 58_{Y1}/58_{Y2}, 58_{Z1}/58_{Z2}) in order to realize catheter navigation in a biological conductor. Alternatively, these orthogonal fields can be decomposed and any pairs of surface electrodes can be driven as dipoles to provide effective electrode triangulation. Likewise, the patch electrodes 58 (or any number of electrodes) could be positioned in any other effective arrangement for driving a current to or sensing a current from an electrode in the heart. For example, multiple electrodes could be placed on the back, sides, and/or belly of patient 14. Additionally, such non-orthogonal methodologies add to the flexibility of the system. For any desired axis, the potentials measured across the roving electrodes resulting from a predetermined set of drive (source-sink) configurations may be combined algebraically to yield the same effective potential as would be obtained by simply driving a uniform current along the orthogonal axes.

Thus, any two of the surface electrodes 58 may be selected as a dipole source and drain with respect to a ground reference, such as belly patch 58_{B}, while the unexcited electrodes measure voltage with respect to the ground reference. The roving electrodes 42 placed in the heart 12 are exposed to the field from navigational currents and are measured with respect to ground, such as belly patch 58_{B}. As previously noted, at least one electrode may be fixed to the interior surface of the heart to form a fixed reference electrode, which is also measured with respect to ground, such as belly patch 58_{B}, and which may be defined as the origin of the coordinate system relative to which electroanatomical mapping system 20 measures positions. Data sets from each of the surface electrodes, the internal electrodes, and the virtual electrodes may all be used to determine the location of the roving electrodes 42 within heart 12.

The measured voltages may be used by electroanatomical mapping system 20 to determine the location in three-dimensional space of the electrodes inside the heart, such as roving electrodes 42 relative to a reference location, such as a fixed intra-cardiac reference electrode. That is, the voltages measured at the fixed intra-cardiac reference electrode may be used to define the origin of a coordinate system, while the voltages measured at roving electrodes 42 may be used to express the location of roving electrodes 42 relative thereto. In some embodiments, the coordinate system is a three-dimensional (x, y, z) Cartesian coordinate system, although other coordinate systems, such as polar, spherical, and cylindrical coordinate systems, are contemplated.

As should be clear from the foregoing discussion, the data used to determine the location of the electrode(s) within the heart is measured while the surface electrode pairs impress an electric field on the heart. The electrode data may also be used to create a respiration compensation value used to improve the raw location data for the electrode locations as described, for example, in U.S. Pat. No. 7,263,397, which is hereby incorporated herein by reference in its entirety. The electrode data may also be used to compensate for changes in the impedance of the body of the patient as described, for example, in U.S. Pat. No. 7,885,707, which is also incorporated herein by reference in its entirety.

Therefore, in one representative embodiment, electroanatomical mapping system 20 first selects a set of surface electrodes 58 and then drives them with current pulses. While the current pulses are being delivered, electrical activity, such as the voltages measured with at least one of the remaining surface electrodes 58 and in vivo electrodes 42, is measured and stored. Compensation for artifacts, such as respiration and/or impedance shifting, may be performed as indicated above.

In aspects of the disclosure, electroanatomical mapping system 20 can be a hybrid system that incorporates both impedance-based and magnetic-based localization capabilities. It is also contemplated, however, that electroanatomical mapping system 20 may be exclusively impedance-based (as described above) or exclusively magnetic field-based.

In some embodiments, electroanatomical mapping system 20 is the EnSite™ X, EnSite™ Velocity™, or EnSite Precision™ electrophysiological mapping and visualization system of Abbott Laboratories. Other systems, however, may be used in connection with the present teachings, including for example the RHYTHMIA HDX™ mapping system of Boston Scientific Corporation (Marlborough, Massachusetts), the CARTO navigation and location system of Biosense Webster, Inc. (Irvine, California), the AURORA® system of Northern Digital Inc. (Waterloo, Ontario), Stereotaxis, Inc.'s NIOBE® Magnetic Navigation System (St. Louis, Missouri), as well as MediGuide™ Technology from Abbott Laboratories.

The localization and mapping systems described in the following patents (all of which are hereby incorporated by reference in their entireties) can also be used with the present invention: U.S. Pat. Nos. 6,990,370; 6,978,168; 6,947,785; 6,939,309; 6,728,562; 6,640,119; 5,983,126; and 5,697,377.

The display device 24, which, as described above, may be part of the electroanatomical mapping system 20 or a separate and distinct component, is provided to convey information to a clinician to assist in, for example, the formation of lesions in the tissue 12. The display device 24 may comprise a conventional computer monitor or other display device.

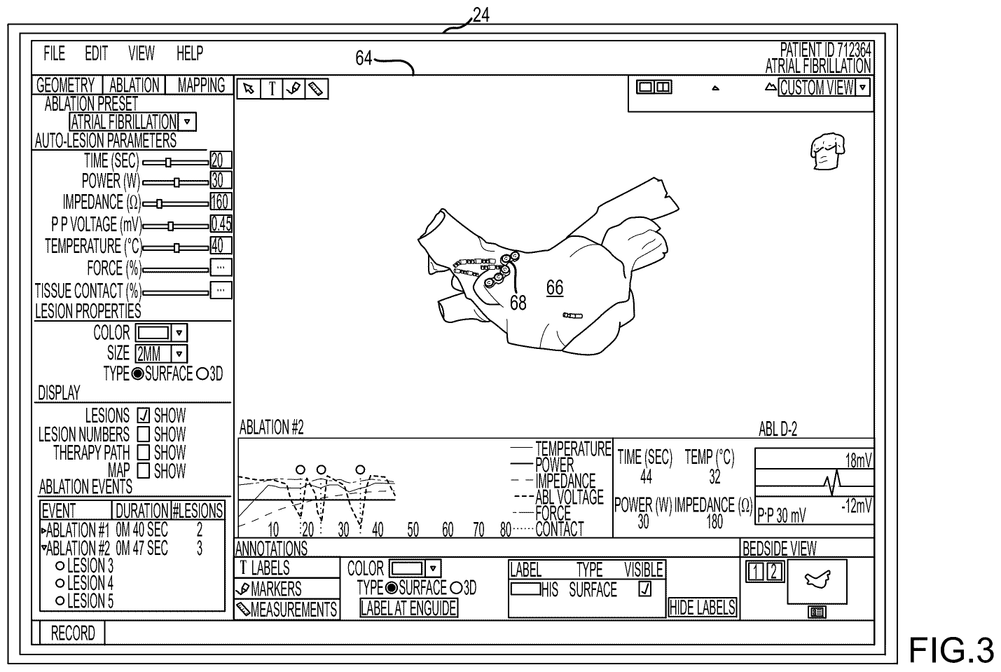

With reference to FIG. 3, display device 24 can present a graphical user interface (GUI) 64 to the clinician. The GUI 64 may include a variety of information including, for example and without limitation, an image or model of the geometry of the tissue 12, EP data associated with the tissue 12, electrocardiograms, ablation data associated with the tissue 12 and/or the ablation generator 52, lesion markers corresponding to lesion segments in the tissue 12, electrocardiogramaps, and images of the catheter 16 and/or positioning electrode 42. Some or all of this information may be displayed separately (e.g., on separate screens) or simultaneously on the same screen (e.g., in one or more windows). GUI 64 may further provide a means by which a clinician may input information or selections relating to various features of the system 10 into ECU 22.

The image or model of the geometry of the tissue 12 (e.g., image/model 66 shown in FIG. 3) may be a two-dimensional image of the tissue 12 (e.g., a cross-section of the heart) or a three-dimensional image of the tissue 12 (e.g., a geometric model of the heart). The image or model 66 may be generated by ECU 22 of electroanatomical mapping system 20, or alternatively, may be generated by another imaging, modeling, or visualization system (e.g., fluoroscopic, computed tomography (CT), magnetic resonance imaging (MRI), direct visualization, or the like) that are communicated to, and therefore acquired by, the ECU 22.

As previously mentioned, the ordinarily-skilled artisan will be familiar with various techniques for creating lesion markers both automatically (e.g., through software implemented on ECU 22) and manually (e.g., via user inputs through GUI 64) in connection with an ablation procedure. Thus, the creation of a set of lesion markers need not be described in detail in order to understand the instant disclosure. By way of example only, however, U.S. Pat. No. 9,204,927, which is hereby incorporated by reference as though fully set forth herein, describes lesion markers that can be automatically created by a diagnostic and/or therapeutic system (such as system 10) based upon the delivery of sufficient ablative energy to the tissue.

Aspects of the disclosure relate to identifying optimized groupings of and interconnections between such lesion markers (and, therefore, the lesion segments represented thereby) and calculating corresponding interlesion distances (that is, calculating optimized interlesion distances). System 10 can therefore include a lesion segment analysis module or processor, which may be implemented via ECU 22 (e.g., in hardware, software, or a combination thereof).

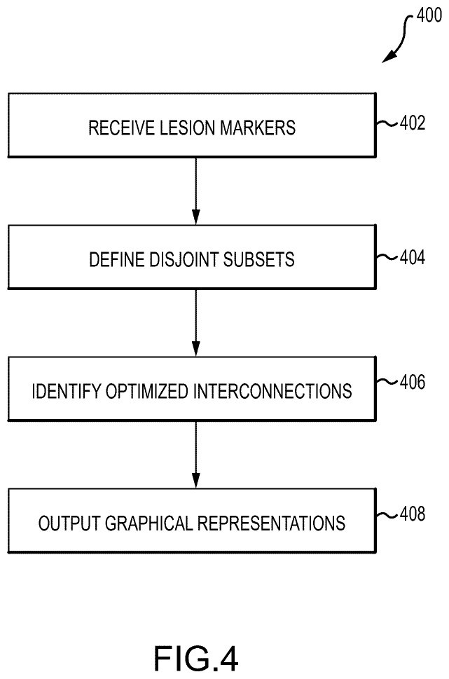

Exemplary methods of optimizing groupings of and interconnections between lesion markers according to aspects of the instant disclosure will be explained with reference to the flowchart 400 of representative steps presented as FIG. 4. In some embodiments, for example, flowchart 400 may represent several exemplary steps that can be carried out by electroanatomical mapping system 20 of FIGS. 1 and 2 (e.g., by ECU 22). It should be understood that the representative steps described below can be hardware-implemented, software-implemented, or implemented in a combination of hardware and software.

In block 402, electroanatomical mapping system 20 receives a plurality of lesion markers. Each lesion marker represents an ablation lesion segment in the tissue of heart 12. Stated differently, each lesion marker is a data point that includes location data (e.g., as measured by electroanatomical mapping system 20) for an ablation lesion segment on heart 12. As discussed above, lesion markers can be created both automatically by electroanatomical mapping system 20 (e.g., when sufficient ablative energy has been applied to heart 12 at a given location to assume that a lesion segment has been created at that location) and manually by a clinician using electroanatomical mapping system 20 (e.g., through GUI 64).

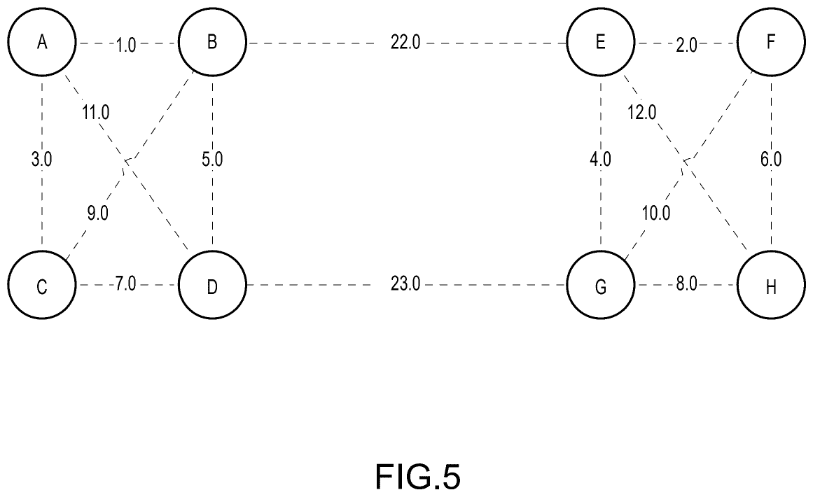

FIG. 5 illustrates a plurality of lesion markers, denoted A-H, such as may be received by electroanatomical mapping system 20 in block 402. FIG. 5 is also annotated with distances (e.g., Euclidean distances) between pairs of lesion markers—for instance, the distance between lesion marker A and lesion marker B is 1.0 mm, while the distance between lesion marker B and lesion marker E is 22.0 mm.

Those of ordinary skill in the art will recognize that FIG. 5 is a simplified, two-dimensional depiction of the plurality of lesion markers received by electroanatomical mapping system 20 in block 402 that is provided solely to aid understanding of the instant disclosure. The teachings herein can, of course, be applied to a much larger set of lesion markers than the eight shown in FIG. 5.

Likewise, the ordinarily-skilled artisan will understand that lesion markers will typically be arranged in three-dimensions, consistent with the geometry of heart 12. Thus, in certain embodiments of the disclosure, the distances between lesion markers may be measured along the surface of heart 12, rather than as the Euclidean distance between lesion markers (though lesion markers will typically be sufficiently close to each other that there will be minimal difference between the two measures, and the use of Euclidean distances may reduce the complexity of, and thus the computational resources required for, the methods described herein).

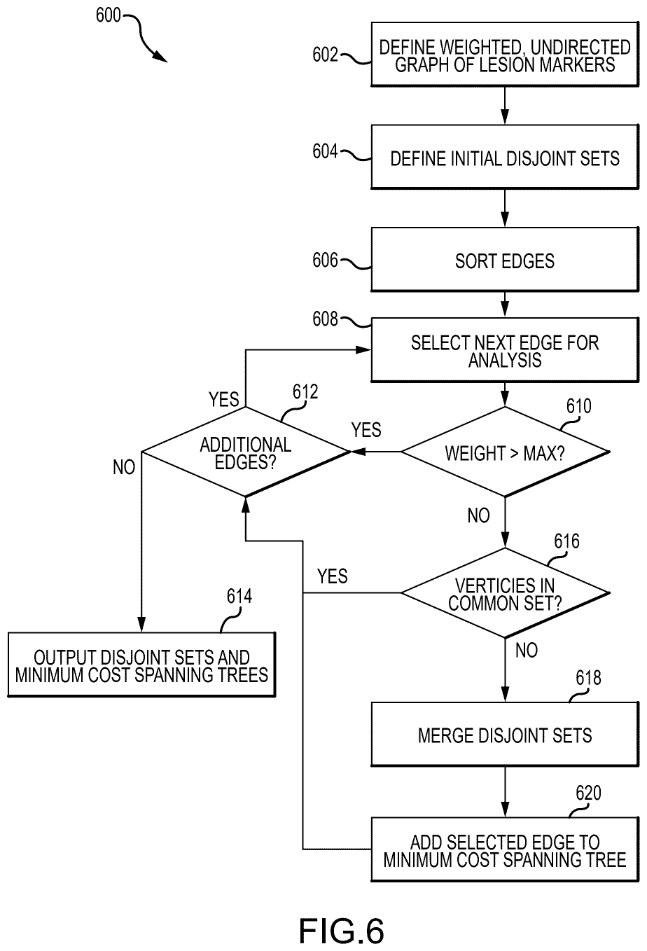

In block 404, electroanatomical mapping system 20 defines one or more disjoint subsets of the plurality of lesion markers. In some embodiments of the disclosure, block 404 can follow the representative steps shown in the flowchart 600 of FIG. 6. More particularly, flowchart 600 of FIG. 6 illustrates exemplary steps that, when executed by electroanatomical mapping system 20 (e.g., by ECU 22), will output one or more disjoint subsets of the plurality of lesion markers as well as a minimum cost spanning tree for each disjoint subset.

In block 602, therefore, electroanatomical mapping system 20 defines a weighted, undirected graph from the plurality of lesion markers received in block 402. The vertices of the graph correspond to the lesion markers, and each weighted edge connects two vertices as shown, for example in FIG. 5. According to aspects of the disclosure, the weight of each edge corresponds to the distance (e.g., the Euclidean distance) between the vertices connected by that edge. In other aspects, the weight of each edge can correspond to a drop in the electrical impedance of the tissue between the vertices connected by that edge.

In block 604, electroanatomical mapping system 20 defines a plurality of initial disjoint subsets, with each initial disjoint subset including exactly one vertex of the graph. In block 606, electroanatomical mapping system 20 sorts the edges of the graph by ascending weight.

Once sorted, the edges are analyzed iteratively, with the next edge to be analyzed selected in block 608. In decision block 610, the weight of the selected edge is compared to a maximum weight threshold. The maximum weight threshold represents a maximum distance between lesion markers that can be regarded as part of the same lesion. In other words, if two lesion markers are further apart from each other than the maximum weight threshold, it can be assumed that they represent lesion segments that are part of discrete lesions. In embodiments of the disclosure, the maximum weight threshold is about 20.0 mm, but it is contemplated that a clinician may set a different value for the maximum weight threshold (e.g., by adjusting a slider or other control on GUI 64).

If the weight of the selected edge exceeds the maximum weight threshold, then analysis of the selected edge stops and the process continues to decision block 612 through the “YES” exit from decision block 610 to determine whether additional edges remain for analysis. If additional edges remain for analysis, another iteration occurs through the “YES” exit from decision block 612. If there are no additional edges to analyze, the process outputs all remaining disjoint subsets and their corresponding minimum cost spanning trees in block 614.

If, on the other hand, the weight of the selected edge is below the maximum weight threshold, analysis proceeds to decision block 616 through the “NO” exit from decision block 610. Decision block 616 analyzes whether the vertices connected by the selected edge are already in the same disjoint subset (referred to herein as a “common disjoint subset”). If so, analysis of the selected edge stops and the process continues to decision block 612, described above, through the “YES” exit from decision block 616.

If, on the other hand, the vertices connected by the selected edge are not already in a common disjoint subset, then the two disjoint subsets respectively containing the two vertices of the selected edge are merged into a combined disjoint subset in block 618 through the “NO” exit from decision block 616. Further, in block 620, the selected edge is added to the minimum cost spanning tree for the combined disjoint subset. Analysis then proceeds to decision block 612, described above.

The process described above can be further understood with reference to FIG. 5. At block 602, electroanatomical mapping system 20 defines a weighted, undirected graph from lesion markers A through H. Each lesion marker A through H becomes a vertex of this graph, and they are interconnected by weighted, undirected edges as shown in dashed line.

In block 604, electroanatomical mapping system 20 defines eight initial disjoint subsets, with each one containing exactly one of the eight vertices of the graph (e.g., {A}, {B}, {C}, {D}, {E}, {F}, {G}, and {H}). The edges are sorted by increasing weight in block 606: AB, EF, AC, EG, BD, FH, CD, GH, BC, FG, AD, EH, BE, and DG. (It should be understood that this list of sorted edges is exemplary, provided for the sake of illustration, and not exhaustive of all the edges that can be made from the aforementioned set of vertices.)

Edge AB is therefore the first edge selected in block 608. Because the weight of edge AB (1.0 mm) is less than the maximum weight threshold (20.0 mm), analysis proceeds through decision block 610 into decision block 616. Likewise, because vertices A and B are not already in a common disjoint subset, they are merged into a combined disjoint subset (e.g., {A, B}) in block 618 and edge AB is added to the corresponding minimum cost spanning tree in block 620.

Additional edges remain for analysis, so the process proceeds through decision block 612 and returns to block 608, where edge EF is selected for analysis. The weight of edge EF (2.0 mm) is less than the maximum weight threshold (20.0 mm), and vertices E and F are not already in a common disjoint subset. Thus, vertices E and F are merged into a combined disjoint subset (e.g., {E, F}) in block 618 and edge EF is added to the corresponding minimum cost spanning tree in block 620.

Additional edges remain for analysis, so similar iterations occur for edges AC, EF, BC, and FH. As those of ordinary skill in the art will appreciate, after edge FH is analyzed, there will be two disjoint subsets: a first disjoint subset containing vertices {A, B, C, D} and a second disjoint subset containing vertices {E, F, G, H}. The minimum cost spanning tree for the first disjoint subset will contain edges AB, AC, and BD, while the minimum cost spanning tree for the second disjoint subset will contain edges EF, EG, and FH.

Still further edges remain for analysis, so the process will once again proceed through decision block 612 and return to block 608, where edge CD is selected for analysis. The weight of edge CD (7.0 mm) is less than the maximum weight threshold (20.0 mm), but vertices C and D are already in a common disjoint subset (e.g., {A, B, C, D}, as described above). Thus, the process immediately returns to decision block 612 without merging two disjoint subsets in block 618 or adding edge CD to the corresponding minimum cost spanning tree in block 620.

Similar iterations occur for edges GH, BC, FG, AD, and EH. That is, there is no merger of disjoint subsets or addition of edges to a minimum cost spanning tree for any of these five edges.

On the other hand, the weight of edge BE (22.0 mm) and the weight of edge DG (23.0 mm) both exceed the maximum weight threshold (20.0 mm). Thus, during analysis of edges BE and DG, the process moves to decision block 612 through the “YES” exit of decision block 610, once again without merging disjoint subsets or adding edge BE or edge DG to a minimum cost spanning tree.

Once the last edge is analyzed, the process exits decision block 612 through the “NO” exit into block 614.

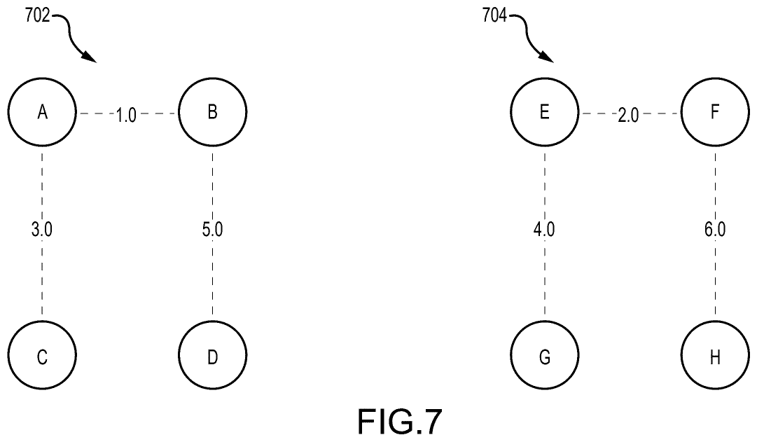

FIG. 7 depicts the two disjoint subsets output in block 614. The first disjoint subset 702 includes vertices (lesion markers) A, B, C, and D and minimum cost spanning tree edges AB, AC, and BD. The second disjoint subset 704 includes vertices (lesion markers) E, F, G, and H and minimum cost spanning tree edges EF, EG, and FH.

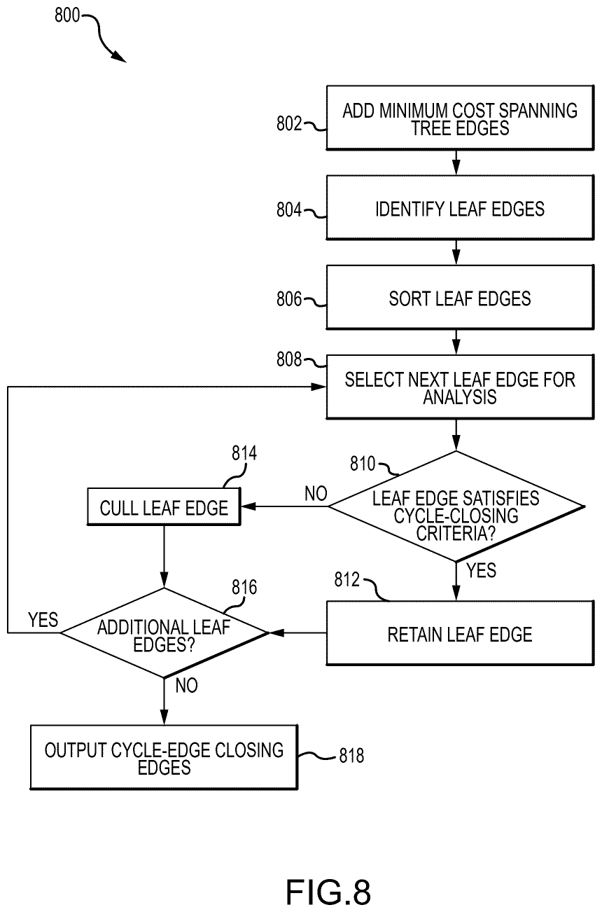

Returning now to FIG. 4, electroanatomical mapping system 20 analyzes each disjoint subset defined in block 404 to identify optimized interconnections between lesion markers in block 406. In some embodiments of the disclosure, block 406 can follow the representative steps shown in the flowchart 800 of FIG. 8 to identify optimized interconnections for a disjoint subset.

In block 802, the edges of the minimum cost spanning tree for the disjoint subset are added to the optimized interconnections. That is, in embodiments of the disclosure, the optimized interconnections for any given disjoint subset include at least the edges in the corresponding minimum cost spanning tree.

The optimized interconnections for a disjoint subset may also include edges that are not included in the corresponding minimum cost spanning tree for the disjoint subset. Because these edges will close cycles when added to the minimum cost spanning tree, they are referred to herein as “cycle-closing edges.”

Not all cycle-closing edges, however, are optimized interconnections. Rather, blocks 804 through 814 illustrate representative steps that may be executed by electroanatomical mapping system 20 to analyze cycle-closing edges and identify those that should be added as optimized interconnections for the disjoint subset.

In block 804, electroanatomical mapping system 20 identifies any leaf edges of the minimum cost spanning tree for the disjoint subset. A leaf is a vertex with a degree of one—that is, a vertex that is part of only a single edge. In turn, leaf edges are edges the interconnect the leaves of the minimum cost spanning tree. To illustrate, consider FIG. 7: vertices C, D, G, and H are leaves, and edges CD, DG, and GH would be leaf edges.

In block 806, electroanatomical mapping system 20 sorts the leaf edges by ascending weight. As described above, the weight of a leaf edge can correspond to the distance (e.g., Euclidean distance) between the vertices of that edge.

Once sorted, the leaf edges are analyzed iteratively, with the next leaf edge to be analyzed selected in block 808. In decision block 810, the selected leaf edge is tested against one or more cycle-closing criteria; various cycle closing criteria are discussed in further detail below. If the selected leaf edge satisfies the cycle-closing criteria (the “YES” exit from decision block 810), it is retained in block 812. Otherwise (the “NO” exit from decision block 810), it is culled in block 814.

In either case, analysis then proceeds to decision block 816, which checks whether additional leaf edges remain for analysis. If so, another iteration occurs through the “YES” exit. If not, the set of cycle-closing edges (i.e., leaf edges that were retained in block 812) is output in block 818 and added to the optimized interconnections for the disjoint subset through the “NO” exit from decision block 816.

As mentioned above, various cycle-closing criteria are contemplated. One suitable cycle-closing criterion is a maximum weight threshold criterion. Similar to the maximum weight threshold discussed above, the maximum weight threshold criterion verifies that the weight of a selected leaf edge is below a maximum value. In embodiments of the disclosure, the maximum weight threshold criterion is 20.0 mm, though it is contemplated that a clinician may set a different value (e.g., through GUI 64).

Referring to FIGS. 5 and 7, leaf edges CD (weight of 7.0 mm) and GH (weight of 8.0 mm) satisfy the maximum weight threshold criterion. Leaf edge DG (weight of 23.0 mm), however, does not, and would be culled in block 814.

Another suitable cycle-closing criterion is a cycle presence criterion. This cycle-closing criterion verifies that both vertices of the selected leaf edge are still leaves. In other words, it verifies that neither vertex of the selected leaf edge was part of a lower-weight leaf edge that was retained cycle during a previous iteration of analysis.

Additional cycle-closing criteria can be defined with reference to the shortest path through the minimum cost spanning tree from one vertex of the selected leaf edge to the other vertex of the selected leaf edge, ensuring that selected leaf edges are only retained to close cycles when the resulting cycle is long enough. In particular, it is desirable for the shortest path through the minimum cost spanning tree to exceed a minimum total weight (e.g., Euclidean distance) and to pass through at least a requisite number of vertices (or, put differently, to include at least a requisite number of edges of the minimum cost spanning tree). The former cycle-closing criterion is referred to herein as a “minimum cost spanning tree path minimum weight criterion” and, in embodiments of the disclosure, it can be set to about 20.0 mm. The latter cycle-closing criterion is referred to herein as a “minimum cost spanning tree path minimum step criterion” and, in embodiments of the disclosure, can be set to about 3. As with other thresholds described herein, of course, it is regarded as within the scope of the instant disclosure to permit a clinician to adjust either or both of these values (e.g., via GUI 64).

Still another suitable cycle-closing criterion is referred to as a “concavity criterion.” Put simply, a concavity criterion ensures that, if the selected leaf edge is retained to close a cycle, the resulting cycle is deep enough. One way to compute concavity is to identify the vertex along the shortest path through the minimum cost spanning tree from one vertex of the selected leaf edge to the other vertex of the selected leaf edge that is furthest (e.g., by Euclidean distance) from the midpoint of the selected leaf edge; this distance is denoted “r.” If r is greater than about 1.5 times the weight of the selected leaf edge, then the selected leaf edge can be considered to satisfy the concavity criterion.

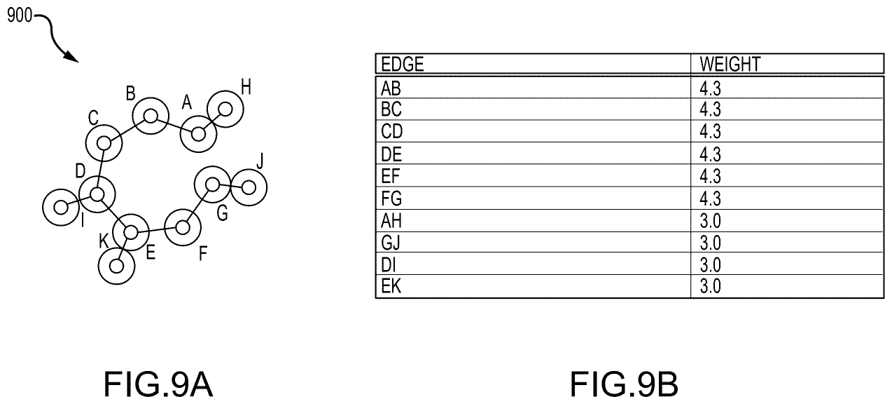

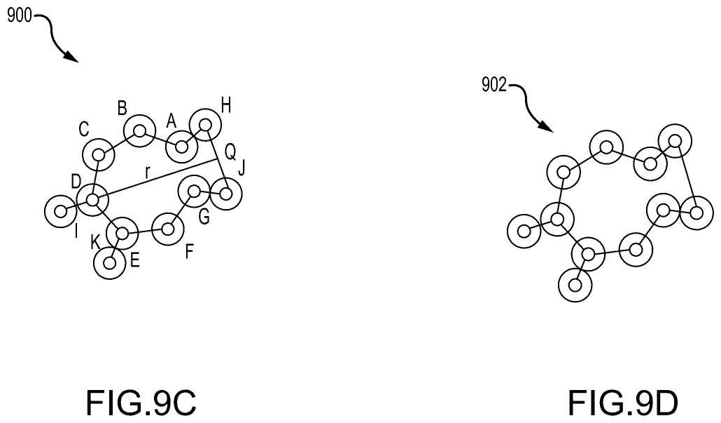

FIGS. 9A through 9D illustrate the application of a minimum cost spanning tree path minimum weight criterion, a minimum cost spanning tree path minimum step criterion, and a concavity criterion. FIG. 9A illustrates a minimum cost spanning tree 900 for a set of vertices (i.e., lesion markers) denoted A through J. Edge weights (e.g., interlesion Euclidean distances, in mm) for minimum cost spanning tree 900 are shown in FIG. 9B.

Consider leaf edge HJ. Assume for purposes of illustration that leaf edge HJ satisfies any applicable maximum weight threshold and cycle presence criteria. Further assume that the weight (e.g., interlesion Euclidean distance, in mm) of leaf edge HJ is 6.9.

The shortest path through minimum cost spanning tree 900 from vertex H to vertex J is H->A->B->C->D->E->F->G->J. The total weight of this path is 31.8, and it includes eight steps. Thus, leaf edge HJ would satisfy both a minimum cost spanning tree path minimum weight criterion of 20.0 and a minimum cost spanning tree path minimum step criterion of 3.

FIG. 9C illustrates the midpoint Q of leaf edge HJ. Assume that vertex D is the furthest vertex from midpoint Q at a distance r of 12.13. Because this exceeds 1.5 times the weight of leaf edge HJ (1.5*6.9=10.35), leaf edge HJ also satisfies a concavity criterion.

Leaf edge HJ can therefore be retained in block 812. The resulting cycle 902 is illustrated in FIG. 9D.

Next consider leaf edge IK. Assume for purposes of illustration that leaf edge IK satisfies any applicable maximum weight threshold and cycle presence criteria.

The shortest path through minimum cost spanning tree 900 from vertex I to vertex K is I->D->E->K. The total weight of this path is 10.3. Thus, leaf edge IK does not satisfy a minimum cost spanning tree path minimum weight criterion of 20.0, and it would be culled in block 814 accordingly.

Referring once again to FIG. 8, in block 808 electroanatomical mapping system 20 can output a graphical representation of the one or more disjoint sets and the one or more optimized interconnections between lesion markers on a graphical representation of the tissue. For example, electroanatomical mapping system 20 can output cycle 902 of FIG. 9D on GUI 64 of FIG. 3.

Although several embodiments have been described above with a certain degree of particularity, those skilled in the art could make numerous alterations to the disclosed embodiments without departing from the spirit or scope of this invention.

For example, the teachings herein can be applied in real time (e.g., during an ablation procedure) or during post-processing (e.g., to data collected during an ablation procedure performed at an earlier time).

As another example, the teachings herein can be applied not only to identifying optimized interconnections and distances between lesion markers, but also between lesion markers and the positions of one or more ablation electrodes during an ablation procedure (e.g., where a subsequent lesion segment may be created).

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A method of identifying optimized interconnections between ablation lesion segments, comprising:
receiving, at an electroanatomical mapping system, a plurality of lesion markers, each lesion marker of the plurality of lesion markers representing an ablation lesion segment in a tissue;
the electroanatomical mapping system defining one or more disjoint subsets of the plurality of lesion markers;
for each disjoint subset, the electroanatomical mapping system defining one or more optimized interconnections between lesion markers included within the respective disjoint subset; and
outputting on a display of the electroanatomical mapping system a graphical representation of the one or more disjoint sets and the one or more optimized interconnections between lesion markers on a graphical representation of the tissue.

2. The method according to claim 1, wherein the electroanatomical mapping system defining the one or more disjoint subsets of the plurality of lesion markers further comprises the electroanatomical mapping system defining a minimum cost spanning tree for each of the one or more disjoint subsets of the plurality of lesion markers.

3. The method according to claim 2, wherein the electroanatomical mapping system defining the minimum cost spanning tree for each of the one or more disjoint subsets of the plurality of lesion markers comprises the electroanatomical mapping system:
defining a weighted, undirected graph including a plurality of vertices and a plurality of weighted edges, wherein the plurality of vertices correspond to the plurality of lesion markers and each weighted edge of the plurality of weighted edges connects a pair of vertices of the plurality of vertices;
sorting the plurality of weighted edges by ascending weight; and
iteratively analyzing each weighted edge of the sorted plurality of weighted edges, at each iteration merging a first disjoint subset containing a first vertex of the pair of vertices connected by the respective weighted edge and a second disjoint subset containing a second vertex of the pair of vertices connected by the respective weighted edge into a combined disjoint subset and adding the respective weighted edge to a minimum spanning tree of the combined disjoint subset when the weight of the respective weighted edge is below a maximum weight threshold and the first vertex and the second vertex are not in a common disjoint subset prior to being merged into the combined disjoint subset.

4. The method according to claim 3, further comprising, prior to iteratively analyzing the sorted plurality of weighted edges, defining a plurality of initial disjoint subsets, each initial disjoint subset including exactly one of the plurality of vertices of the graph.

5. The method according to claim 3, wherein the weight of each weighted edge comprises a distance between the respective pair of vertices connected by the respective weighted edge.

6. The method according to claim 5, wherein the distance comprises a Euclidean distance.

7. The method according to claim 2, wherein the one or more optimized interconnections between lesion markers included within the respective disjoint subset comprises a plurality of edges within the minimum cost spanning tree for the respective disjoint subset.

8. The method according to claim 7, wherein the one or more optimized interconnections between lesion markers included within the respective disjoint subset further comprises one or more cycle-closing edges not included within the minimum cost spanning tree for the respective disjoint subset.

9. The method according to claim 8, wherein the electroanatomical mapping system identifies the one or more cycle-closing edges not included within the minimum cost spanning tree for the respective disjoint subset according to a series of steps comprising:
defining a plurality of leaf edges of the minimum cost spanning tree for the respective disjoint subset; and
identifying a subset of the plurality of leaf edges that satisfy one or more cycle-closing criteria; and
defining the subset of the plurality of leaf edges that satisfy the one or more cycle-closing criteria as the one or more cycle-closing edges not included within the minimum cost spanning tree.

10. The method according to claim 9, wherein identifying the subset of the plurality of leaf edges that satisfy the one or more cycle-closing criteria comprises:
assigning a weight to each leaf edge of the plurality of leaf edges;
sorting the plurality of leaf edges by ascending weight; and
iteratively analyzing each leaf edge of the sorted plurality of leaf edges with respect to each of the one or more cycle-closing criteria, at each iteration culling the respective leaf edge from the plurality of leaf edges when the respective leaf edge does not satisfy a cycle-closing criterion of the one or more cycle-closing criteria, thereby identifying the subset of the plurality of leaf edges that satisfy the one or more cycle-closing criteria.

11. The method according to claim 9, wherein the one or more cycle-closing criteria comprises one or more of:
a maximum weight threshold criterion;
a cycle presence criterion;
a minimum cost spanning tree path minimum weight criterion;
a minimum cost spanning tree path minimum step criterion; and
a concavity criterion.

12. An electroanatomical mapping system, comprising:
a display; and
a lesion segment analysis processor configured to:
receive as input a plurality of lesion markers, each lesion marker of the plurality of lesion markers representing an ablation lesion segment in a tissue;
define one or more disjoint subsets of the plurality of lesion markers and, for each disjoint subset, define one or more optimized interconnections between lesion markers included within the respective disjoint subset; and
output on the display a graphical representation of the one or more disjoint subsets and the one or more optimized interconnections between lesion markers on a graphical representation of the tissue.

13. The electroanatomical mapping system according to claim 12, wherein the lesion segment analysis processor is further configured to define a minimum cost spanning tree for each of the one or more disjoint subsets of the plurality of lesion markers.

14. The electroanatomical mapping system according to claim 13, wherein the lesion segment analysis processor is configured to define the minimum cost spanning tree for each of the one or more disjoint subsets of the plurality of lesion markers by executing a series of steps comprising:
defining a weighted, undirected graph including a plurality of vertices and a plurality of weighted edges, wherein the plurality of vertices correspond to the plurality of lesion markers and each weighted edge of the plurality of weighted edges connects a pair of vertices of the plurality of vertices;
sorting the plurality of weighted edges by ascending weight; and
iteratively analyzing each weighted edge of the sorted plurality of weighted edges, at each iteration merging a first disjoint subset containing a first vertex of the pair of vertices connected by the respective weighted edge and a second disjoint subset containing a second vertex of the pair of vertices connected by the respective weighted edge into a combined disjoint subset and adding the respective weighted edge to a minimum spanning tree of the combined disjoint subset when the weight of the respective weighted edge is below a maximum weight threshold and the first vertex and the second vertex are not in a common disjoint subset prior to being merged into the combined disjoint subset.

15. The electroanatomical mapping system according to claim 14, wherein the weight of each weighted edge comprises a distance between the respective pair of vertices connected by the respective weighted edge.

16. The electroanatomical mapping system according to claim 13, wherein the one or more optimized interconnections between lesion markers included within the respective disjoint subset comprise a plurality of edges within the minimum cost spanning tree for the respective disjoint subset.

17. The electroanatomical mapping system according to claim 16, wherein the one or more optimized interconnections between lesion markers included within the respective disjoint subset further comprises one or more cycle-closing edges not included within the minimum cost spanning tree for the respective disjoint subset.

18. The electroanatomical mapping system according to claim 17, wherein the lesion segment analysis processor is configured to identify the one or more cycle-closing edges by executing a series of steps comprising:
defining a plurality of leaf edges of the minimum cost spanning tree for the respective disjoint subset;
identifying a subset of the plurality of leaf edges that satisfy one or more cycle-closing criteria; and
defining the subset of the plurality of leaf edges that satisfy the one or more cycle-closing criteria as the one or more cycle-closing edges,
wherein the one or more cycle-closing criteria comprises one or more of:
a maximum weight threshold criterion;
a cycle presence criterion;
a minimum cost spanning tree path minimum weight criterion;
a minimum cost spanning tree path minimum step criterion; and
a concavity criterion.

19. A method of identifying optimized interconnections between ablation lesion segments, comprising:
receiving, at an electroanatomical mapping system, a plurality of lesion makers, each lesion marker of the plurality of lesion markers representing an ablation lesion segment in a tissue;
the electroanatomical mapping system defining one or more disjoint subsets of the plurality of lesion markers;
for each disjoint subset, the electroanatomical mapping system defining one or more optimized interconnections between lesion markers included within the respective disjoint subset, wherein the one or more optimized interconnections between lesion markers included within the respective disjoint subset comprises:
a plurality of edges included in a minimum cost spanning tree for the respective disjoint subset; and
a plurality of cycle-closing edges that close cycles within the minimum cost spanning tree for the respective disjoint subset; and
outputting on a display of the electroanatomical mapping system a graphical representation of the one or more disjoint subsets and the one or more optimized interconnections between lesion markers on a graphical representation of the tissue.

20. The method according to claim 19, wherein the plurality of cycle-closing edges are leaf edges of the minimum cost spanning tree for the respective disjoint subset that satisfy one or more cycle-closing criteria, the one or more cycle-closing criteria comprising one or more of:
a maximum weight threshold criterion;
a cycle presence criterion;
a minimum cost spanning tree path minimum weight criterion;
a minimum cost spanning tree path minimum step criterion; and
a concavity criterion.
